(19) United States
(12) Patent Application Publication
(10) Pub. No.: US 2008/0233580 A1
(43) Pub. Date: Sep. 25, 2008

(54) Transgenic animal model

(75) Inventors: Wanjiang Han, Hauterive (CH); Jutta Schueller, Koeln (DE)

Correspondence Address: BUCHANAN, INGERSOLL & ROONEY PC, POST OFFICE BOX 1404, ALEXANDRIA, VA, 22313-1404, US

(73) Assignee: Philip Morris USA Inc., Richmond, VA (US)

(21) Appl. No.: 12/004,490

(22) Filed: Dec. 21, 2007

(30) Foreign Application Priority Data
Dec. 21, 2006 (EP) 06026563.4

(51) Int. Cl. A01K 67/027; C12N 15/85; C12Q 1/68

(52) U.S. Cl. 435/6; 800/13; 800/21; 435/320.1

(57) ABSTRACT

The present invention is related to a transgenic, non-human animal, particularly a transgenic rodent, but especially a transgenic mouse model which allows for the simultaneous, tissue-specific and temporally-controlled regulation of transgene expression and can be used as a tool to investigate the consecutive steps involved in initiation and progression of certain diseases such as cancer, but particularly lung cancer.

### CROSS-REFERENCE TO RELATED APPLICATION

This application claims priority under 35 U.S.C. §119 to European Application No. 06026563.4, filed Dec. 21, 2006, the entire content of which is hereby incorporated by reference.

### INCORPORATION-BY-REFERENCE OF SEQUENCE LISTING OR TABLE

The material in the accompanying sequence listing is hereby incorporated by reference into this application.

The present invention is related to a transgenic, non-human animal, particularly a transgenic rodent, which allows for the simultaneous, tissue-specific and temporally-controlled regulation of transgene expression. Such model can be used as a tool to investigate the consecutive steps involved in initiation and progression of certain diseases such as cancer, but particularly lung cancer.

Transgenic non-human animal models such as transgenic mouse models have been irreplaceable tools for the study of the molecular and physiological processes involved in certain human disease states, particularly those involved in oncogenicity. Whereas the gene disruption and gene replacement strategies used to develop conventional transgenic mouse models are appropriate to create null mutants or gain-of-function mutants, neither is ideal to model diseases with polygenic etiologies and environmental influences such as, for example, cancer as an “acquired” disease. Such known strategies create germline mutations. As a consequence, the potential to initiate a cascade of secondary responses during the earliest stages of development is substantial. These responses, even if phenotypically silent in the unstressed organism, may considerably alter the behavior to additional challenges in the adult mouse. Although this information, in itself, may be of interest, rendering a transgene silent during development and inducing its expression in the adult provides a potentially more suitable environment which is not complicated by potential developmental perturbations. Moreover, most cancers are sporadic, and carcinogenesis is often tissue or spatially specific. The conventional transgenic mouse carries constitutive transgenes in the complete animal, which creates a very different microenvironment than is found during initiation of sporadic cancers in which a few tumor cells are surrounded by many normal cells that may keep the incipient cancer cell in check by cell-cell contact or paracrine signaling.

Epidemiological data have clearly indicated that cigarette smoke is causally associated with the development of lung cancer. Past molecular biology studies have revealed the complex molecular alterations in lung cancer. It is known that perturbations of the integrity of integrated signaling networks, positively or negatively regulating various cellular processes to maintain homeostasis of the lung, lead to the progression of lung cancer. As with all types of cancer, transformation from a normal lung cell to a malignant lung cancer cell is the result of many combinations of events. Though carcinogenesis of cigarette smoke is believed to be a multistage process, there is little agreement as to which steps are truly required for cancer to develop. The cause and effect relationship between certain genetic or epigenetic aberrations and carcinogenesis is often unclear. This is complicated by the fact that little is known about the early events of cigarette smoke-induced carcinogenesis. Furthermore, both the cell type of origin of lung cancer and the complex, even “paradoxical”, role that the microenvironment plays in tumorigenicity are unknown.

Recent strategies for the development of transgenic mouse models for lung cancers have evolved from targeting transgenes in a constitutive mode to regulating the expression and ablation of genes in the lung in an inducible mode. This allows for the control of gene expression in a spatial- and tissue-specific manner, thereby providing a better platform for further investigation of the molecular basis of tumorigenesis (reviewed by Kwak et al., 2004). The most widely used systems in creating the conditional transgenic mouse are the Cre recombinase of P1 bacteriophage and the Flp recombinase of Saccharomyces cerevisiae yeast, which behave quite similarly to each other. The recombinase enzyme Cre (or Flp) promotes recombination via recognition of a 34-bp asymmetric polynucleotide termed loxP (or fft). Depending on the relative orientation of the loxP sites, Cre may catalyze excision (same orientation) or insertion (opposite orientation) of the DNA segments located between these sites. The known approach to achieve inducible gene targeting involves two types of transgenic mouse lines. The first transgenic mouse line bears the target gene (or gene segment) flanked by two loxP sites in the same orientation and positioned in such a way that it does not prevent normal gene activity (floxed gene). The second transgenic mouse line contains a transgene expressing Cre recombinase. When these two mouse lines are crossed, depending on the promoter and regulatory sequences present in the transgene, the floxed gene is deleted and a mutation is created in particular cells or tissues. It is important to note that two requirements must be fulfilled for conditional gene targeting with the Cre/loxP (or Flp/frt) system. Firstly, the floxed allele must be created in such a way that it is still functional, and secondly, targeting of Cre expression must be tightly controlled. Depending on the floxed DNA sequence, the Cre/loxP system can also be used to create insertion. To this end, conditional transgenes can be introduced not only in a given type of cell but also at a given time in development. These conditional models can be targeted to cancer, since it is a sporadic, that is acquired disease. Therefore, a genetic change can be introduced at any stage of the development of the mouse model. However, in addition to the fact that cancer is often an acquired disease and is subject to the sophisticated complication of the microenvironment, tobacco smoke carcinogenesis shows extra features. Epidemiological data showing that smokers, 15 to 25 years after smoke cessation, face almost the same low risk of developing lung cancer as the non-smoker strongly suggest a “reversibility” or dosage effect of cigarette smoke. Moreover, the insult imposed by cigarette smoke is often intermittent with respect to its mode of action. Several conditional transgenic mouse models for lung cancer have been developed by intranasally or intratracheally administering the adeno-cre virus, a recombinant adenovirus expressing Cre-recombinase (Jackson et al., 2001; Meuwissen et al., 2001). However, the suppression or activation of the targeted genes in this way is irreversible and becomes constitutive after the administration, which does not reflect the intermittent nature of cigarette smoke exposure or the observed reversibility of lung cancer in smokers following cessation. These points raise the question of relevance of the known conventional and conditional transgenic mice as proper disease models for cigarette smoke-induced lung tumorigenicity. In other words, at present there is no proper transgenic mouse model for studying cigarette smoke-induced lung cancer or other smoke-induced diseases.

In summary, an ideal or pertinent non-human animal model for studying cigarette smoke-induced diseases should be able to capture: 1) the acquired nature of the diseases, 2) the complication of the microenvironment, 3) the intermittent characteristics of cigarette smoke exposure, and 4) the “reversible” nature of the carcinogenic process in smokers following cessation.

The present invention now provides such a non-human animal model which possesses the above mentioned advantageous capabilities. In particular, the invention provides a transgenic, non-human animal comprising stably integrated into its genome a first and a second expression cassette, wherein said second expression cassette comprises a polynucleotide encoding an effector polypeptide under the control of a tissue-specific promoter. The expression of said (second) effector polypeptide is activated by the expression product of the first expression cassette, which expression product is encoded by a polynucleotide under the control of an inducible promoter.

According to all aspects and embodiments of the present invention, the preferred transgenic, non-human animal is a transgenic rodent. Particularly preferred is a transgenic mouse.

In particular, the present invention provides a transgenic, non-human animal comprising stably integrated into its genome a first expression cassette and a second expression cassette, wherein said second expression cassette comprises a second polynucleotide encoding a second effector polypeptide under the control of a tissue-specific promoter and, optionally, a further nucleotide sequence, which blocks expression of the second effector polypeptide such that no expression of the second effector polynucleotide occurs from said tissue-specific promoter in the non-induced state, and wherein said first expression cassette comprises a first polynucleotide encoding a first effector polypeptide under the control of an inducible promoter, which, upon induction by an inducing agent or composition and expression of the first effector polypeptide, activates expression of the second effector polypeptide, for example by removing the block from the second expression cassette.

In a specific embodiment of the invention, a transgenic, non-human animal, is provided, wherein the first expression cassette and the second expression cassette are stably integrated in the genome of at least one somatic cell of said transgenic, non-human animal.

In another specific embodiment of the invention, a transgenic, non-human animal is provided, wherein the first and second expression cassette are stably integrated in the genome of at least one germline cell of said transgenic, non-human animal.

Optionally, the second expression cassette comprises a further nucleotide sequence, which blocks expression of the second effector polynucleotide. The blocking nucleotide sequence comprises one or more stop codons or a polyadenylation sequence or a reporter gene or any other nucleotide sequence capable of blocking expression of the second effector polypeptide. Advantageously it is located in the expression cassette such that expression of the effector polypeptide is blocked. Preferably it is located upstream of the nucleotide sequence encoding the second effector polypeptide, more preferably between the promoter and the encoding polynucleotide.

In a specific embodiment of the invention, the first polynucleotide encoding the first effector polypeptide comprised in the first expression cassette under control of an inducible promoter is a recombinase encoding polynucleotide and the blocking sequence is flanked by short nucleotide sequences comprising recognition sites of the recombinase protein.

In particular, the first effector polypeptide is a Cre recombinase or an Flp recombinase and the recombinase recognition sequences flanking the blocking nucleotide sequence, particularly the polyA sequence or stop codon or reporter gene are loxP and frt recognition sequences, respectively.

In a specific embodiment of the invention, the inducible promoter controlling the expression of the first effector polypeptide in the first expression cassette is an on/off-type promoter which is strongly induced and provides essentially no background activity. In particular, induction of said promoter is dose dependent and compound or composition dependent and thus allows to fine tune gene expression levels and durations by modulating the dose and the nature of the inducing agent.

In a specific aspect of the invention an inducible promoter is provided which is strongly induced in the presence of environmental toxicants, for example, nicotine and polycyclic aromatic hydrocarbons (PAH) such as benzo(a)pyrene (BaP), and chlorinated dioxins and furans such as tetrachlorodibenzo-p-dioxin (TCDD), and beta-naphthoflavone (BNF), some of which are present in tobacco smoke, and the like, individually or in different combinations of one or more of said constituents.

In a specific embodiment of the invention, the inducible promoter controlling the expression of the effector polypeptide in the first expression cassette is a promoter controlling expression of a cytochrome P450 mono-oxygenase, but particularly expression of the cyp1A1 gene. Other inducible promoters may also be used within the scope of the present invention for controlling expression of the first effector polypeptide such as, for example, without however being limited thereto, the promoter of stress responsive gene hsp70.1, promoters of metallothionine genes and other cyp-type promoters.

In another aspect of the invention, the tissue-specific promoter controlling expression of the second effector polypeptide comprised in the second expression cassette is a tissue specific promoter, particularly a lung tissue specific promoter, more preferably a promoter which controls expression of lung tissue-specific proteins, most preferably a protein that is specifically expressed in nonciliated bronchial epithelial cells (Clara cells) in respiratory and terminal bronchioles such as, for example, the Clara cell 10 protein. Particularly preferred is the CC10 protein promoter.

In another embodiment of the invention, the lung tissue-specific promoter is a promoter which controls expression of a lung tissue-specific protein, more particularly a protein that is specifically expressed in alveolar epithelial cells such as, for example, the surfactant protein A/C. Such protein is a lung tissue-specific protein, which modulates a number of immune cell functions, including cell proliferation, cytokine production, the expression of cell surface markers, and the generation of oxidative activity. In still another embodiment of the invention, the lung tissue specific promoter is a promoter which controls expression of lung tissue-specific proteins in both type I and type II lung epithelial cells such as, for example, the RAIG1.

Other lung-tissue-specific promoters that may also be used within the scope of the present invention include, for example, a promoter selected from the group consisting of thyroid transcription factor-2 (TTF1), the promoter of aquaporin 5, the promoter of T1 alpha and the promoter of retinoic acid-induced gene 1 (RAIG1). Thyroid transcription factor-1 (TTF-1, product of the Nk×2.1 gene) is essential for branching morphogenesis of the lung and enhances expression of surfactant proteins by alveolar type II cells. T1 alpha is a differentiation gene of lung alveolar epithelial type I cells. Aquaporin-5 (AQP5) is a water channel protein expressed on the apical surface of alveolar epithelial type I cells. RAIG1 is a gene specifically expressed in lung epithelial cells of both type I and type II. In a further aspect of the invention, the transgenic, non-human animal according to the present invention and described herein before comprises stably integrated in its genome a gene of interest (GOI) the expression of which is modulated by the second effector polypeptide.

In particular, a transgenic, non-human animal is provided comprising stably integrated into its genome a first expression cassette and a second expression cassette, wherein said second expression cassette comprises a polynucleotide encoding a second effector polypeptide under the control of a tissue-specific promoter, the expression of which is activated by the expression product of the first expression cassette which expression product is encoded by a polynucleotide under the control of an inducible promoter and wherein said transgenic, non-human animal comprises in its genome a gene of interest (GOI), which is integrated in the animal's genome in an active state or actively expressed from the genome, but becomes inactivated upon expression of the second effector polypeptide.

In another embodiment, the invention provides a transgenic, non-human animal, particularly a transgenic rodent, comprising stably integrated into its genome a first and a second expression cassette, wherein said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state, and wherein said first expression cassette comprises a polynucleotide encoding a first effector polypeptide under the control of an inducible promoter, which, upon induction by an inducing agent or composition and expression of the first effector polypeptide, activates expression of the second effector polypeptide, for example by removing the block from the second expression cassette, and wherein said transgenic non-human animal, particularly said transgenic mouse, comprises in its genome a gene of interest (GOI), which is integrated into the animal's genome such that it is actively expressed from said genome in the non-induced state, but becomes inactivated upon expression of the second effector polypeptide.

The gene of interest may either be a native gene which is present in the genome of the transgenic, non-human animal, or, in the alternative, a transgene that has been introduced into the animal's genome artificially through recombinant DNA techniques. The gene of interest may, for example, be a tumor suppressor gene such as the tumor suppressor gene k-Ras 12 (Jackson et al., 2001), Rb1 or Trp53 (Meuwissen et al., 2001), or any other gene that can be suitably used within the scope of the present invention, which gene is operably integrated into the genome of the transgenic, non-human animal and actively expressed from the genome in the non-induced state, but becomes blocked or inactivated upon expression of the second effector polypeptide.

In a specific embodiment of the invention, the second effector polypeptide is a recombinase and the gene of interest in the genome of the transgenic, non-human animal is flanked by short nucleotide sequences comprising recognition sites of a recombinase protein which do not interfere with the normal expression of the gene of interest. In particular, the effector polypeptide is a Cre recombinase or an Flp recombinase and the recombinase recognition sequences flanking the gene of interest (GOI) are loxP and frt recognition sequences, respectively.

In another aspect of the invention, the gene of interest (GOI) is integrated in the genome of the transgenic, non-human animal in an inactive state or not actively expressed from the genome.

In particular, a transgenic, non-human animal is provided comprising stably integrated into its genome a first expression cassette and a second expression cassette, wherein said second expression cassette comprises a polynucleotide encoding a second effector polypeptide under the control of a tissue-specific promoter, the expression of which is activated by the expression product of the first expression cassette which expression product is encoded by a polynucleotide under the control of an inducible promoter and wherein said transgenic, non-human animal comprises in its genome a gene of interest (GOI), which is integrated in the animal's genome in an inactive state or is not actively expressed from the genome, but becomes activated upon expression of the second effector polypeptide.

In another embodiment, the invention provides a transgenic, non-human animal comprising stably integrated into its genome a first and a second expression cassette, wherein said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state, and wherein said first expression cassette comprises a polynucleotide encoding a first effector polypeptide under the control of an inducible promoter, which, upon induction by an inducing agent or composition and expression of a first effector polypeptide, activates expression of the second effector polypeptide, for example by removing the block from the second expression cassette, and wherein said transgenic, non-human animal comprises stably integrated in its genome a gene of interest (GOI), which is integrated in the animal's genome such that it is not actively expressed from said genome in the non-induced state, but becomes activated upon expression of the second effector polypeptide.

The blocking polynucleotide may comprise a stop codon or a polyadenylation sequence or a reporter gene and is particularly located in the expression cassette such that expression of the effector polypeptide is blocked, but especially between the promoter and the encoding polynucleotide.

The gene of interest may either be a native gene which is present in the non-human animal's genome or, in the alternative, a transgene that has been introduced into the genome artificially through recombinant DNA techniques. The inactive gene of interest (GOI) is deblocked or activated upon expression of the second effector polypeptide such that expression of the gene of interest is caused.

The gene of interest may, for example, be a tumor susceptibility gene such as a tumor susceptibility gene selected from the group consisting of a lung adenoma susceptibility gene 1 (Las-1) and Kirstin rat sarcoma oncogene 2 (Kras2) or any other gene that can be suitably used within the scope of the present invention. Further comprised by the present invention is a vector molecule comprising a first expression cassette and a second expression cassette as part of the same vector molecule, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter, the expression of which is activated by the expression product of the first expression cassette.

In particular, the invention relates to a vector molecule, wherein the second expression cassette comprises a further polynucleotide, which blocks expression of the second effector polypeptide.

In one embodiment, the invention provides a vector molecule comprising a first expression cassette and a second expression cassette as part of the same vector molecule, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state. The expression of the second effector polypeptide is activated by the expression product of the first expression cassette.

The optional further nucleotide sequence, which blocks expression of the second effector polypeptide such that no expression of the effector nucleotide occurs from the tissue-specific promoter in the non-induced state, may be a nucleotide sequence comprising one or more stop codons, a polyadenylation (polyA) sequence, a reporter gene or any other sequence capable of blocking expression of the second effector polypeptide and is particularly located in the expression cassette such that expression of the second effector polypeptide is blocked. Preferably the nucleotide sequence is located upstream of the polynucleotide encoding the second effector polypeptide, more preferably between the promoter and the encoding nucleotide sequence.

Also comprised is a vector molecule, wherein the inducible promoter controlling the expression of the effector polypeptide in the first expression cassette is an on/off-type promoter which is strongly induced and provides essentially no background activity and is dependent on the dose and the nature of the inducing compound or composition, particularly on cigarette smoke or at least one of its constituents, particularly a promoter controlling expression of a cytochrome P450 mono-oxygenase, but especially a promoter controlling expression of the cyp1A1 gene.

In a further embodiment, a vector molecule according to the present invention is provided, wherein the tissue-specific promoter is a lung tissue-specific promoter, particularly a promoter which controls expression of lung tissue-specific proteins, especially proteins that are specifically expressed in nonciliated bronchial epithelial cells (Clara cells) in respiratory and terminal bronchioles such as, for example, the Clara cell 10 protein, particularly the CC10 protein promoter. Alternatively, the vector molecule according to the present invention may comprise a lung tissue-specific promoter which controls expression of lung tissue-specific proteins that are specifically expressed in alveolar epithelial cells such as, for example, the surfactant protein A/C.

In a further aspect of the invention, the vector molecule according to the present invention comprises a first effector polypeptide, which is a recombinase, particularly a Cre recombinase or a Flp recombinase and/or a second effector polypeptide, which is a recombinase, particularly a Cre recombinase or a Flp recombinase.

Further provided is a vector molecule according to the invention comprising an expression cassette comprising a gene of interest targeted with recombinase recognition sequences, particular a floxed targeted transgene of interest.

In one embodiment of the invention, a vector molecule is provided wherein the gene of interest is a tumor suppressor gene such as p53, Arf, Dmp1 and Rb or a tumor susceptibility gene such as k-Ras 12, WT1, TSG101, etc., and wherein the gene is flanked by Cre recognition sequences.

In yet another embodiment of the invention, a vector molecule is provided comprising a first expression cassette, a second expression cassette and a third expression cassette as part of the same vector molecule, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state, the expression of which is activated by the expression product of the first expression cassette, and said third expression cassette comprises a gene of interest targeted with recombinase recognition sequences, particular a floxed targeted transgene of interest particularly a tumor suppressor gene such as p53, Arf, Dmp1 and Rb or a tumor susceptibility gene such as k-Ras 12, WT1, TSG101, etc., particularly flanked by Cre recognition sequences.

In yet another embodiment, the present invention provides a method of producing a transgenic non-human animal, particularly a transgenic rodent, but especially a transgenic mouse according to the present invention comprising transfecting a target animal with a vector molecule according to the invention and as described herein before. In still another embodiment of the invention, a method is provided of evaluating the carcinogenic potential of an agent or a composition in a specific tissue of the animal when applied intermittently comprising: (i) contacting the transgenic, non-human animal according to the invention and as described herein before with the agent or composition to be evaluated; (ii) blocking or inactivating expression of a gene of interest thus causing genetic aberrations within the cells of a specific tissue of the animal, (iii) identifying and determining said genetic aberrations; (iv) comparing the number of genetically altered cells in a sample from the treated animal with the number of genetically altered cells in a sample from an untreated transgenic animal or transgenic animal treated with a control agent, wherein the difference in the number of transformed cells in the treated animal, relative to the number of transformed cells in the absence of treatment or treatment with a control agent, indicates the carcinogenic potential of the test compound.

Also comprised by the present invention is a method for evaluating the reversibility of the carcinogenic process induced by an agent or composition comprising: (i) contacting the transgenic, non-human animal according to the invention and as described herein before with the agent or composition to be evaluated intermittently according to a defined time schedule; (ii) blocking or inactivating expression of a gene of interest thus causing genetic aberrations within the cells of a specific tissue of the animal, (iii) identifying and determining said genetic aberrations; (iv) discontinuing contacting the transgenic animal with the agent or composition to be evaluated, comparing the number of genetically altered cells in a sample from the intermittently treated animal with the number of altered cells in a sample from a animal at a given time after treatment had been discontinued, and (v) determining reversibility of the carcinogenic process.

Many diseases which originate from some type of insult to a particular tissue or cell of the organism such as, for example, cigarette smoke-induced diseases, particularly cigarette smoke-induced cancer have the nature of “acquiredness”. This type of disease is referred to as a sporadic disease as opposed to a familial disease. Therefore, the development of the disease is affected by the complication of the microenvironment, which can either stop, slow down, or speed up the disease. Moreover, in case of certain cigarette smoking-related diseases epidemiological evidence clearly illustrates that these diseases can be considered as being “reversible”.

Therefore, the present invention provides an animal model which reflects both, the dose delivered and the intermittent mode of the exposure. In particular, the transgenic, non-human animal according to the present invention allows for a tissue-specific, particularly lung tissue-specific and temporally controlled expression of a gene of interest caused by the intermittent exposure of the model animal to a disease-inducing compound or composition, particularly by exposure to cigarette smoke followed by a transgene activation or inactivation of the gene of interest.

In a specific embodiment of the invention, a transgenic, non-human animal is provided wherein an expression cassette comprising a tissue-specific promoter, particularly a lung tissue-specific promoter such as, for example, a SP-A/C or CC10 promoter is combined with another expression cassette comprising an inducible promoter, particularly a promoter induced by environmental toxicants such as, for example, cigarette smoke or one of more of its constituents, particularly a cyp1A1 promoter, wherein both promoters drive the expression of an effector gene, particularly a recombinase gene such as, for example, a cre or an flp recombinase gene, which provides a feasible and powerful tool for investigating the consecutive steps involved in initiation and progression of certain diseases such as cancer, but particularly lung cancer.

A lung tissue-specific promoter that can be suitably used within the non-human animal model system according to the invention is the SP-A/C promoter or the CC10 promoter, respectively. Surfactant protein A/C is a lung tissue-specific protein, which modulates a number of immune cell functions, including cell proliferation, cytokine production, the expression of cell surface markers, and the generation of oxidative activity. It may also participate in the adaptive immune response. Due to its lung tissue-specificity, surfactant protein A/C promoter has been used in several studies to introduce genetic alterations in lung cells (Harrod et al., 1998; Wilmott et al., 1998). Clara cell 10 protein is the predominant product from nonciliated bronchial epithelial cells (Clara cells) in respiratory and terminal bronchioles in the lung. Since approximately 50-70% of epithelial cells in the trachea, bronchia, and bronchioles (pulmonary conducting airways) of mice are Clara cells, the Clara cell CC10 protein promoter has also been used to express transgenes in a lung tissue-specific manner in several studies (Temann et al., 1998; Fisher et al., 2001).

An inducible promoter that can be suitably used within the model system according to the invention is the cyp1A1 promoter. The cyp1A1 gene is a gene from the cytochrome P450 mono-oxygenase system. The cytochrome P450 mono-oxygenase system represents a major defense against chemical challenge from the environment, constituting part of an adaptive response mounted by an organism following exposure to harmful agents, such as cigarette smoke. They also participate in a variety of essential “house-keeping” functions, such as biosynthesis of steroid hormones and fatty acid oxidation. Cytochrome P450s, however, are also able to catalyse the activation of certain compounds to toxic products. These enzymes are highly regulated by chemical inducing agents. Of particular interest in this regard is the cytochrome P-450 cyp1A1 gene. This gene is not constitutively expressed but is highly inducible upon exposure to cigarette smoke and/or the constituents thereof and environmental toxicants such as, nicotine, polycyclic aromatic hydrocarbons (PAH), tetrachlorodibenzo-p-dioxin (TCDD), and beta-naphthoflavone (BNF), etc. A significant advantage of the cyp1A1 system is that there is essentially no background activity in the lung, and it provides the ability to fine tune gene expression levels and durations by modulating the dose and the nature of the inducing agent. Indeed several studies have shown that the cyp1A1 promoter in transgenic mice is an exquisitely sensitive on-off system for cell specific gene regulation (Campbell et al., 1996; Smith et al., 1995; Ryding et al., 2001). Not only is the expression of cyp1A1 induced by exposure to cigarette smoke, it is also induced by various environmental toxicants. This opens up the possibility of developing this type of conditional transgenic mice for toxicological testing and environmental monitoring as well.

In particular, the invention provides a transgenic, non-human animal comprising stably integrated into the mouse genome a first and a second expression cassette, wherein said second expression cassette comprises a polynucleotide encoding an effector polypeptide under the control of a tissue-specific promoter, particularly a lung tissue specific promoter such as, for example the Clara cell CC10 protein promoter or the surfactant protein A/C promoter or a promoter that controls expression of lung tissue specific proteins in both type I and type II lung epithelial cells such as, for example, the RAIG1, the expression of which is activated by the expression product of the first expression cassette which expression product is encoded by a polynucleotide under the control of an inducible promoter, particularly a promoter that is inducible by cigarette smoke or by one or more of its constituents such as, for example, the cyp1A1 promoter.

In another embodiment, the invention provides a transgenic, non-human animal, particularly a mouse comprising stably integrated into the mouse genome a first and a second expression cassette, wherein said second expression cassette comprises a polynucleotide encoding an effector polypeptide, particularly a recombinase such as, for example, a Cre recombinase or a Flp recombinase, under the control of a tissue-specific promoter, particularly a lung tissue-specific promoter such as, for example the Clara cell CC10 protein promoter or the surfactant protein A/C promoter, the expression of which is activated by the expression product of the first expression cassette which expression product is an effector polypeptide, particularly a recombinase such as, for example, a Cre recombinase or a Flp recombinase, encoded by a polynucleotide under the control of an inducible promoter, particularly a promoter that is inducible by cigarette smoke or by one or more of its constituents such as, for example, the cyp1A1 promoter and a further polynucleotide, which blocks expression of the second effector polypeptide in the non-induced state, particularly a polynucleotide comprising a stop codon or a polyadenylation sequence or a reporter gene which is located in the expression cassette such that expression of the effector polypeptide is blocked, but especially between the promoter and the encoding polynucleotide and is flanked by recombinase recognition sequences such as, for example, loxP and frt recognition sequences.

In a further embodiment, the transgenic, non-human animal according to the present invention and as described herein before comprises in its genome a gene of interest (GOI), for example a tumor suppressor gene such as the tumor suppressor gene k-Ras 12, Rb1, Trp53, the WT1 or TSLC1 or a tumor suppressor gene located on the short arm of chromosome 3, in an area designated as 3p21.3 such as, for example the Ras association domain family 1 (RASSFI) including RASSFIA and RASSFIC, or any other gene that can be suitably used within the scope of the present invention, which is integrated in the animals genome such that it is actively expressed from the animals genome in the non-induced state, but becomes inactivated upon expression of the second effector polypeptide.

In a specific embodiment of the invention, the second effector polypeptide is a recombinase, particularly a Cre recombinase or a Flp recombinase, but especially a FLP recombinase and the gene of interest in the animal's genome is flanked by short polynucleotides comprising recognition sites of the recombinase protein which do not interfere with the normal expression of the gene of interest, particularly loxP or frt recognition sequences, but especially frit recognition sequences.

The dual promoter system according to the present invention comprising an inducible promoter, particularly a promoter inducible by cigarette smoke or one or more of its constituents and a tissue-specific promoter, particularly a lung tissue specific promoter, allows for control of the initiation of carcinogenesis in a tissue-specific and controlled manner, particularly in a lung tissue-specific and cigarette smoke controlled manner.

In one embodiment, a method for producing a transgenic non-human animal model, particularly a mouse model, is provided comprising
(a) providing a first expression cassette and a second expression cassette, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter, the expression of which is activated by the expression product of the first expression cassette;
(b) introducing said first and second expression cassette into the genome of an animal, particularly a mouse by
   (i) introducing said first and second expression cassette into a fertilized mouse egg or a blastocyst of an embryo;
      transferring the fertilized egg containing said expression cassettes to the oviduct of a pseudopregnant female mouse wherein the mouse becomes pregnant; or, in the alternative
      transplanting the embryo containing said expression cassettes into a pseudopregnant mouse and allowing said blastocyst to develop to term;
(c) allowing the pregnant female mouse to deliver progeny mice; and
(d) selecting from the progeny mice those which have stably incorporated into the genome the first and second expression cassette.

In one embodiment, a method for producing a transgenic non-human animal model, particularly a mouse model, is provided comprising
(a) providing a first expression cassette and a second expression cassette, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter, the expression of which is activated by the expression product of the first expression cassette;
(b) transfecting a population of mouse embryonic stem cells with a first expression cassette and a second expression cassette;
(c) identifying a mouse embryonic stem cell having said first and second expression cassette integrated into its genome by homologous recombination;
(d) inserting said cell into a mouse embryo; and
(d) allowing the resulting embryo to grow thereby producing the transgenic mouse according to the invention and as described herein before.

In one embodiment, the first expression cassette and the second expression cassette according to the invention and as described herein before may each be present on a separate vector molecule and transferred to the animal to be transfected together or sequentially.

In another embodiment of the invention the first and the second expression cassettes according to the invention and as described herein before are both comprised within a single vector molecule.

In one embodiment, a method for producing a transgenic non-human animal model, particularly a mouse model, is provided comprising
(a) providing an expression cassette comprising a gene of interest targeted with recombinase recognition sequences, particular a floxed targeted transgene of interest such as floxed k-Ras 12 or floxed p53 and Rb, wherein the gene is flanked by Cre recognition sequences;
(b) introducing said expression cassette into the genome of an animal, particularly a mouse by
   (i) introducing introducing said first and second expression cassette into a fertilized mouse egg or a blastocyst of an embryo;
      transferring the fertilized egg containing said expression cassette to the oviduct of a pseudopregnant female mouse wherein the mouse becomes pregnant; or, in the alternative
      transplanting the embryo containing said expression cassette into a pseudopregnant mouse and allowing said blastocyst to develop to term;
(c) allowing the pregnant female mouse to deliver progeny mice; and
(d) selecting from the progeny mice those which have stably incorporated into the genome the gene of interest targeted with recombinase recognition sequences.

In one embodiment, a method for producing a transgenic non-human animal model, particularly a mouse model, is provided comprising
(a) providing an expression cassette comprising a gene of interest targeted with recombinase recognition sequences, particular a floxed targeted transgene of interest such as floxed k-Ras 12 or floxed p53 and Rb, wherein the gene is flanked by Cre recognition sequences;
(b) transfecting a population of mouse embryonic stem cells with said expression cassette;
(c) identifying a mouse embryonic stem cell having said expression cassette integrated into its genome by homologous recombination;
(d) inserting said cell into a mouse embryo; and
(e) allowing the resulting embryo to grow thereby producing the transgenic mouse according to the invention and as described herein before having stably incorporated into the genome the gene of interest targeted with recombinase recognition sequences.

In a specific embodiment of the invention, a first mouse A, the transactivator mouse, is provided in which the sporadic expression of a recombinase, particularly a recombinase Cre is lung tissue-specific and regulated by a cigarette smoke-inducible promoter, particularly a cyp1A1 promoter. This mouse A is then crossed with a second mouse B comprising in its genome a gene of interest targeted with recombinase recognition sequences, particular a floxed targeted transgene such as floxed k-Ras 12 (Jackson et al., 2001) or floxed p53 and Rb (Meuwissen et al., 2001), wherein the gene is flanked by Cre recognition sequences.

The methods for producing mouse A and B used in this cross are well known in the art and described, for example, in Nagy et al., 2002: Manipulating the mouse embryos: A laboratory manual; Cold Spring Harbor Laboratory Press, 3rd edition.

The offspring of said cross which carries the dual promoter system and the floxed targeted genes can be used to study molecular mechanisms of cigarette smoke-induced diseases. Initiation of carcinogenesis in these mice can be controlled with respect to time by cigarette smoke exposure. Since recombinase Flp (and hence Cre as well) will only be sporadically or temporally induced by cigarette smoke, the genetic mutation or aberration will occur only in several proximal cells at each time of exposure and after cessation of the exposure gene expression may be restored, which reflects the intermittent exposure of cigarette smoke in smokers. Since surfactant protein A/C or Clara cell 10 protein is only expressed in lung tissue, the expression of recombinase Cre and hence the genetic aberration is tissue-specifically controlled in the lung tissues. The transactivator mouse can also be used to generate transgenic mice in which gene expression is knocked down or down regulated with temporal and lung tissue specificity.

In one embodiment of the invention a method is therefore provided for evaluating the carcinogenic potential of an agent or a composition in a specific tissue upon intermittent exposure to said agent or composition comprising: (i) intermittently exposing a transgenic non-human animal, particularly a transgenic rodent, but especially a transgenic mouse according to the present invention and as described herein before to an agent or composition to be evaluated thus inducing sporadically or temporally an effector polypeptide in a tissue-specific manner; (ii) blocking or inactivating expression of a gene of interest thus causing genetic aberrations within the cells of a specific tissue of the animal, (iii) identifying and determining said genetic aberrations; (iv) comparing the number of genetically altered cells in a sample from the treated animal with the number of genetically altered cells in a sample from an untreated transgenic animal or transgenic animal treated with a control agent, wherein the difference in the number of transformed cells in the treated animal, relative to the number of transformed cells in the absence of treatment or treatment with a control agent, indicates the carcinogenic potential of the test compound.

In a specific aspect of the invention, a method is provided for evaluating the carcinogenic potential of cigarette smoke or of at least one constituent thereof in lung tissue upon intermittent exposure to cigarette smoke or to at least one constituent thereof comprising: (i) intermittently exposing a transgenic non-human animal, particularly a transgenic rodent, but especially a transgenic mouse according to the present invention and as described herein before to cigarette smoke or the at least one constituent thereof to be evaluated thus inducing sporadically or temporally an effector polypeptide in a lung tissue-specific manner; (ii) blocking or inactivating expression of a gene of interest thus causing genetic aberrations within the cells of a specific tissue of the animal, (iii) identifying and determining said genetic aberrations; (iv) comparing the number of genetically altered cells in a sample from the treated animal with the number of genetically altered cells in a sample from an untreated transgenic animal or transgenic animal treated with a control agent, wherein the difference in the number of transformed cells in the treated animal, relative to the number of transformed cells in the absence of treatment or treatment with a control agent, indicates the carcinogenic potential of cigarette smoke or of at least one constituent thereof.

In another aspect of the invention, a method is provided for evaluating the reversibility of the carcinogenic process induced by an agent or composition comprising: (i) intermittently exposing according to a defined time schedule the transgenic non-human animal, particularly a transgenic rodent, but especially a transgenic mouse according to the present invention and as described herein before to the agent or composition to be evaluated; (ii) blocking or inactivating expression of a gene of interest thus causing genetic aberrations within the cells of a specific tissue of the animal, (iii) identifying and determining said genetic aberrations; (iv) discontinuing exposure of the transgenic animal to the agent or composition to be evaluated, (iv) comparing the number of genetically altered cells in a sample from the intermittently treated test animal with the number of altered cells in a sample from an animal at a given time after treatment had been discontinued, and (iv) determining reversibility of the carcinogenic process.

In still another embodiment, the invention relates to a method for evaluating the reversibility of the carcinogenic process induced by cigarette smoke or at least one constituent thereof in lung tissue comprising: (i) intermittently exposing according to a defined time schedule the transgenic non-human animal, particularly the transgenic rodent, but especially the transgenic mouse according to the present invention and as described herein before to cigarette smoke or to at least one constituent thereof to be evaluated; (ii) blocking or inactivating expression of a gene of interest thus causing genetic aberrations within the cells of a specific tissue of the animal, (iii) identifying and determining said genetic aberrations; (iv) discontinuing exposure of the transgenic animal to cigarette smoke or at least one constituent thereof to be evaluated, (iv) comparing the number of genetically altered cells in a sample from the intermittently treated test animal with the number of altered cells in a sample from an animal at a given time after treatment had been discontinued, and (iv) determining reversibility of the carcinogenic process.

The transgenic non-human animal model according to the present invention can thus be used for investigate the significance of certain genetic or epigenetic aberrations in cigarette smoke-induced carcinogenesis or other diseases in a spatial- and temporal-specific manner.

### BRIEF DESCRIPTION OF THE DRAWINGS

FIGS. 1(A) and 1(B) are schematic diagrams of vector constructs according to embodiments described herein.

### EXAMPLES

### Example 1

### Construction of Expression Cassette 1 for Inducible Expression of Flp

For the inducible expression of Flp, a vector is constructed using standard rDNA techniques such as those described in Campbell et al, 1996 or Ryding et al, 2001. The flp transgene is operably linked with the inducible cyp1a1 promoter and the metallothionine (MT-1) polyadenylation sequences such that the coding sequence of Flp recombinase is expressed under the control of the inducible promoter.

Flp cDNA is amplified by PCR from plasmid 706-FLP (Gene Bridges) and cloned into pBS185 (Invitrogen) replacing the Cre recombinase sequence to produce pBS185-FLP. PBS185-FLP carrying the CMV promoter, the FLP coding sequence and the mouse MT-1 polyadenylation site is further digested by Spel and Xhol to eliminate the CMV promoter sequence. The 11.5-kb fragment of rat Cyp1A1 promoter containing regions exon 1 and part of exon 2 is isolated from pAHIr1-LacZ and cloned into pBS185-FLP. The 14.4-kb Cyp1A1-Flp transgene fragment is generated by HincII and KpnI digestion of pBS185-Cyp1A1-Flp vector. Transgenic Cyp1A1-Flp animals are generated by pronuclei microinjections of fertilized oocytes (C57BL6 F1 animals) with Cyp1A1-Flp transgene. The resulting offspring is genotyped by PCR on genomic DNA prepared from tail biopsies.

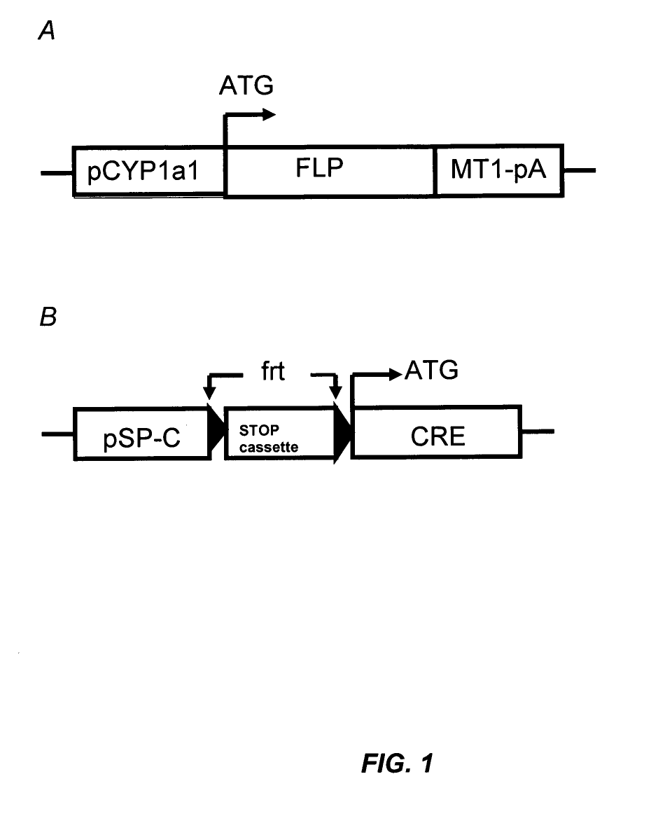

A schematic diagram of the vector construct is given in FIG. 1(A).

### Example 2

### Construction of Expression Cassette 2 for Conditional Expression of Cre

For the conditional expression of Cre in lung tissue a lung tissue-specific promoter SP-C is fused to three MT-1 polyadenylation sequences which are flanked by Flp targeting sequences frt followed by a Cre recombinase coding sequence using standard rDNA techniques such as described, for example, in Harrod et al, 1998 or Wilmott et al, 1998 or Bertin et al, 2005.

pBS185 is digested by Spel and Xhol to eliminate the CMV promoter sequence. The 3.7 kb human SP-C promoter is amplified and cloned into pBS185 to produce SP-C-Cre vector. A 105 bp oligonucleotide containing two frt sites in the same orientation (upper case) encompassing three stop codon sequence (lower case) (5′-GMGTTCCTATTCCGMGTTCCT ATTCTATTCTCTAGAAAGTATAGGMCTTCtgatagtaaGMGTTCCTATTCCGMGTTCCT ATTCTATTCTCTAGAAAGTATAGGMCTTC-3′) (SEQ ID NO: 1) and the complementary oligo are synthesized, annealed and blunt-end cloned into the SP-C-Cre vector. The SPC-frt-STOP-frt-Cre vector is linearized by HincII and KpnI digestion, and the 7.6-kb fragment containing SPC promoter, frt flanked stop codon sequence, Cre recombinase coding sequence and the moust MT-1 polyadenylation site are isolated, purified and injected into the fertilized oocytes (C57BL6 F1 animals). The offspring is genotyped by PCR on genomic DNA prepared from tail biopsies.

A schematic diagram of the vector construct is given in FIG. 1(B).

The pSP-C promoter in FIG. 1B can be the promoter of SP-A, that of Clara cell 10 protein, that of thyroid transcription factor 1 (TTF-1), that of T1 alpha, that of aquaporin 5, or that of RAIG-1 gene.

### Example 3

### Generation of Transgenic Mouse A

Expression cassettes 1 and 2 are transfected into a first mouse A, the transactivator mouse, simultaneously or consecutively using standard transfection technology. Mouse A provides for sporadic expression of a recombinase, particularly a recombinase Cre in a lung tissue-specific manner and regulated by a cigarette smoke-inducible promoter, particularly a cyp1A1 promoter.

In this configuration, the Flp expression, which can be induced by cigarette smoke, results in the deletion of the three MT-1 polyadenylation sequences and thus the expression of Cre in lung tissue.

Transgenic mouse A is obtained from crosses of Cp1A1-Flp and SP-C-frt-STOP-frt-Cre mice. In this configuration, the Flp expression, which can be induced by cigarette smoke, results in the deletion of the stop cassette sequences and thus the expression of Cre in lung tissue.

### Example 4

### Transfection of a Vector Comprising a Gene of Interest (GOI) into Mouse B

A vector comprising the gene of interest such as k-Ras 12 (Jackson et al., 2001) or p53 and Rb (Meuwissen et al., 2001) is constructed as described for example in Kwak et al (2004) and transfected into a second mouse B using standard transfection technology to produce a mouse comprising in its genome a gene of interest targeted with recombinase recognition sequences, particular a floxed targeted transgene such as floxed k-Ras 12 or floxed p53 and Rb, wherein the gene is flanked by Cre recognition sequences.

### Example 5

### Crossing of Mouse A X Mouse B

Mouse A is crossed with mouse B to produce offspring. The offspring of said cross carries the dual promoter system and the floxed targeted gene of interest and can be used to study molecular mechanisms of cigarette smoke-induced diseases. Initiation of carcinogenesis in these mice can be controlled with respect to time by cigarette smoke exposure. Since recombinase Flp (and hence Cre as well) will only be sporadically or temporally induced by cigarette smoke, the genetic mutation or aberration will occur only in several proximal cells at each time of exposure and after cessation of the exposure gene expression may be restored, which reflects the intermittent exposure of cigarette smoke in smokers. Since surfactant protein A/C or Clara cell 10 protein is only expressed in lung tissue, the expression of recombinase Cre and hence the genetic aberration is tissue-specifically controlled in the lung tissues.

All of the above-mentioned references are herein incorporated by reference in their entirety to the same extent as if each individual reference was specifically and individually indicated to be incorporated herein by reference in its entirety.

While the invention has been described with reference to preferred embodiments, it is to be understood that variations and modifications may be resorted to as will be apparent to those skilled in the art. Such variations and modifications are to be considered within the purview and scope of the invention as defined by the claims appended hereto.

### REFERENCE LIST

Bertin et al. 2005, Transgenic Research 14, 645-654
Campbell et al. 1996; Regulation of the CYP1A1 promoter in transgenic mice: an exquisitely sensitive on-off system for cell specific gene regulation. J Cell Sci. 109 (Pt 11): 2619-2625
Fisher et al., 2001, Induction and apoptotic regression of lung adenocarcinomas by regulation of a K-Ras transgene in the presence and absence of tumor suppressor genes, Genes Dev. 15:3249-3262
Harrod et al., 1998; Lung-specific expression of adenovirus E3-14.7K in transgenic mice attenuates adenoviral vector-mediated lung inflammation and enhances transgene expression. Hum Gene Ther. 9:1885-1898
Jackson et al., 2001; Analysis of lung tumor initiation and progression using conditional expression of oncogenic K-ras. Genes Dev. 15:3243-3243
Kwak et al., 2004; Genetically engineered mouse models for lung cancer, Annue Rev. Physiol. 66:647-663
Meuwissen et al., 2001; Mouse model for lung tumorigenesis through Cre/lox controlled sporadic activation of the K-Ras oncogene. Oncogene. 20:6551-6558
Ryding et al., 2001; Conditional transgenic technologies. J Endocrinol. 171:1-14
Smith et al., 1995; Cytochrome P450 1A1 promoter as a genetic switch for the regulatable and physiological expression of a plasma protein in transgenic mice. Proc Natl Acad Sci USA 92:11926-11930
Temann et al., 1998; Expression of interleukin 9 in the lungs of transgenic mice causes airway inflammation, mast cell hyperplasia, and bronchial hyperresponsiveness. J Exp Med. 188:1307-1320
Wilmott et al., 1998; Generation of a transgenic mouse with lung-specific overexpression of the human interleukin-1 receptor antagonist protein. Am J Respir Cell Mol Biol. 18:429-434

1. A transgenic animal comprising stably integrated into the animal genome a first and a second expression cassette,
wherein said second expression cassette comprises a polynucleotide encoding a second effector polypeptide under the control of a tissue-specific promoter, the expression of which is activated by the expression product of the first expression cassette which expression product is encoded by a polynucleotide under the control of an inducible promoter.

2. The transgenic animal according to claim 1
wherein said second expression cassette comprises a further nucleotide sequence which blocks expression of the second effector polypeptide such that no expression of the effector nucleotide occurs from this promoter in the non-induced state, and wherein said first expression cassette comprises a polynucleotide encoding a first effector polypeptide under the control of an inducible promoter, which, upon induction by an inducing agent or composition and expression of the first effector polypeptide, removes the block from the second expression cassette and activates expression of the second effector polypeptide.

3. The transgenic animal according to claim 1 said transgenic animal comprises in its genome a gene of interest (GOI), which is integrated in the animal genome in an active state and/or actively expressed from the genome, but becomes inactivated upon expression of the second effector polypeptide.

4. The transgenic animal according to claim 2 said transgenic animal comprises in its genome a gene of interest (GOI), which is integrated in the animal genome such that it is actively expressed from the animal genome in the non-induced state, but becomes inactivated upon expression of the second effector polypeptide.

5. The transgenic animal according to claim 1, wherein the inducible promoter controlling the expression of the effector polypeptide in the first expression cassette is an on/off-type promoter which is strongly induced and provides essentially no background activity, which promoter is dependent on the dose and the nature of the inducing compound or composition.

6. A transgenic animal according to claim 1, wherein the tissue specific promoter is a lung tissue-specific promoter.

7. A transgenic animal according to claim 1, wherein the first and the second effector polypeptide are recombinases.

8. A vector molecule comprising a first and a second expression cassette as part of the same vector molecule, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state, the expression of which is activated by the expression product of the first expression cassette.

9. The vector molecule according to claim 8, wherein
a) the inducible promoter controlling the expression of the effector polypeptide in the first expression cassette is an on/off-type promoter which is strongly induced and provides essentially no background activity, which promoter is dependent on the dose and the nature of the inducing compound or composition; and
b) the tissue specific promoter is a lung tissue-specific promoter.

10. A method of producing a transgenic animal comprising transfecting a target animal with a vector molecule comprising a first and a second expression cassette as part of the same vector molecule, wherein said first expression cassette comprises a polynucleotide encoding a first effector polypeptide under the control of an inducible promoter, which, upon induction by an inducing agent or composition expresses a first effector polypeptide, and said second expression cassette comprises a polynucleotide encoding a second effector polypeptide under the control of a tissue-specific promoter and, optionally, a further nucleotide sequence, which blocks expression of the second effector polypeptide such that no expression of the effector nucleotide occurs from this promoter in the non-induced state, the expression of which is activated by the expression product of the first expression cassette.

11. A method of evaluating the carcinogenic potential of an agent or a composition in a specific tissue of an animal when applied intermittently comprising:
(i) contacting the transgenic animal of claim 1 with the agent or composition to be evaluated;
(ii) blocking or inactivating expression of a gene of interest thus causing genetic aberrations within the cells of a specific tissue of the animal,
(iii) identifying and determining said genetic aberrations; and
(iv) comparing the number of genetically altered cells in a sample from the treated animal with the number of genetically altered cells in a sample from an untreated transgenic animal or transgenic animal treated with a control agent, wherein the difference in the number of transformed cells in the treated animal, relative to the number of transformed cells in the absence of treatment or treatment with a control agent, indicates the carcinogenic potential of the test compound.

12. A method for evaluating the reversibility of the carcinogenic process induced by an agent or composition comprising:
(i) contacting the transgenic animal of claim 1 with the agent or composition to be evaluated intermittently according to a defined time schedule;
(ii) blocking or inactivating expression of a gene of interest thus causing genetic aberrations within the cells of a specific tissue of the animal;
(iii) identifying and determining said genetic aberrations;
(iv) discontinuing contacting the transgenic animal with the agent or composition to be evaluated;
(v) comparing the number of genetically altered cells in a sample from the intermittently treated animal with the number of altered cells in a sample from a animal at a given time after treatment had been discontinued; and
(vi) determining reversibility of the carcinogenic process.

13. The transgenic animal according to claim 5,
wherein said inducible promoter controlling the expression of the effector polypeptide in the first expression cassette is selected from the group consisting of a promoter controlling expression of a cytochrome P450 mono-oxygenase and a promoter controlling expression of the cyp1 A1 gene; and
wherein said inducing compound or composition comprises cigarette smoke, nicotine, a polycyclic aromatic hydrocarbon, a chlorinated dioxin, and/or a furan.

14. The transgenic animal according to claim 13, wherein said inducing compound or composition comprises benzo(a)pyrene (BaP), tetrachlorodibenzo-p-dioxin, and/or beta-naphthoflavone (BNF).

15. The transgenic animal according to claim 6, wherein said lung tissue-specific promoter which controls expression of a protein in nonciliated bronchial epithelial cells (Clara cells), in alveolar epithelial cells, and/or in both type I and type II lung epithelial cells.

16. The transgenic animal according to claim 15, wherein the protein whose expression is controlled by the lung-tissue specific promoter is the Clara cell 10 protein, the surfactant protein A/C, and/or RAIG1.

17. The transgenic animal according to claim 7, wherein said recombinases comprise a Cre recombinase and/or a Flp recombinase.

18. The vector molecule according to claim 9,
wherein said inducible promoter controlling the expression of the effector polypeptide in the first expression cassette is selected from the group consisting of a promoter controlling expression of a cytochrome P450 mono-oxygenase and a promoter controlling expression of the cyp1A1 gene; and
wherein said inducing compound or composition comprises cigarette smoke, nicotine, a polycyclic aromatic hydrocarbon, a chlorinated dioxin, and/or a furan.

19. The vector molecule according to claim 18, wherein said inducing compound or composition comprises benzo(a)pyrene (BaP), tetrachlorodibenzo-p-dioxin, and/or beta-naphthoflavone (BNF).

20. The vector molecule according to claim 9, wherein said lung tissue-specific promoter which controls expression of a protein in nonciliated bronchial epithelial cells (Clara cells), in alveolar epithelial cells, and/or in both type I and type II lung epithelial cells.

21. The vector molecule according to claim 20, wherein the protein whose expression is controlled by the lung-tissue specific promoter is the Clara cell 10 protein, the surfactant protein A/C, and/or RAIG1.


SEQUENCE LISTING (1 sequence)

SEQ ID NO: 1 - DNA, 115 bp, Artificial Sequence
  Description of Artificial Sequence Synthetic oligonucleotide
gaagttcctattccgaagttcctattctattctctagaaagtataggaacttctgatagtaagaagttcctattccgaagttcctattctattctctagaaagtataggaacttc